(19) United States
(12) Patent Application Publication
(10) Pub. No.: US 2013/0190631 A1
(43) Pub. Date: Jul. 25, 2013

(54) APPARATUS FOR DETECTING RESPIRATORY SINUS ARRHYTHMIA

(71) Applicant: BIOTRONIK SE & CO. KG, Berlin (DE)

(72) Inventor: Thomas Doerr, Berlin (DE)

(73) Assignee: BIOTRONIK SE & CO. KG, Berlin (DE)

(21) Appl. No.: 13/738,997

(22) Filed: Jan. 11, 2013

Related U.S. Application Data
(60) Provisional application No. 61/588,187, filed on Jan. 19, 2012.

(51) Int. Cl. A61B 5/0205; A61N 1/362

(52) U.S. Cl. CPC A61B 5/0205; A61N 1/3621
USPC 600/484; 607/14

(57) ABSTRACT

At least one embodiment of the invention relates to an implantable electronic device, comprising a heart rhythm detection unit, a respiratory dynamics detection unit, an evaluation unit and at least one connection to at least two electrode poles. The at least two electrode poles are connected to the heart rhythm detection unit, and the heart rhythm detection unit is configured to supply a heart rhythm signal representing the heart rhythm as an output signal. The at least two electrode poles are connected to a respiratory dynamics determination unit, and the respiratory dynamics detection unit is configured to supply a respiratory dynamics signal representing the respiratory dynamics as the output signal. Moreover, the heart rhythm detection unit and the respiratory dynamics detection unit are connected to the evaluation unit, and the evaluation unit is configured to evaluate the heart rhythm signal in conjunction a temporally associated respective respiratory dynamics signals.

This application claims the benefit of U.S. Provisional Patent Application 61/588,187 filed on 19 Jan. 2012, the specification of which is hereby incorporated herein by reference.

### BACKGROUND OF THE INVENTION

1. Field of the Invention

At lest one embodiment of the invention relates to an implantable electronic device or device module for cardiac function diagnostics.

2. Description of the Related Art

Implantable systems, such as cardiac pacemakers and implantable defibrillators (ICD, CRT-D), already now detect a number of diagnostic parameters, which are made available to the physician for follow-up monitoring of the patient. These are primarily rhythmological parameters, which are derived from the heart rate. In addition, respiratory parameters and activity parameters can be captured.

Heart rate variability parameters are used above all for predicting imminent cardiac dysrhythmia and/or cardiac decompensation (see, for example, U.S. Pat. No. 7,181,270).

The aforementioned parameters have so far not been able to show a sufficiently specific result with respect to cardiac follow-up monitoring, in particular for predicting imminent arrhythmia or cardiac decompensation. Thus, further parameters are required to detect the autonomous control of the heart rhythm.

### BRIEF SUMMARY OF THE INVENTION

It is a feature of at least one embodiment of the invention to provide a device, or parts of a device, that allows or that allow different and/or improved cardiac function diagnostics.

This feature is achieved according to at least one embodiment of the invention by an implantable electronic device or device module, comprising a heart rhythm detection unit, a respiratory dynamics detection unit, an evaluation unit and at least one connection for at least two electrode poles for sensing electrical potentials. The at least two electrode poles are connected or are to be connected to the heart rhythm detection unit, and the heart rhythm detection unit is configured to supply a heart rhythm signal representing a heart rhythm from a signal to be captured via the at least two electrode poles as the output signal. To this end, the at least two electrode poles are connected or are to be connected to the respiratory dynamics determination unit, and the respiratory dynamics determination unit is configured to supply a respiratory dynamics signal representing respiratory dynamics from a signal to be captured via the at least two electrode poles as the output signal. Moreover, the heart rhythm detection unit and the respiratory dynamics detection unit are connected to the evaluation unit, and the evaluation unit is configured to evaluate a respective heart rhythm signal in conjunction with a temporally associated respective respiratory dynamics signal and to generate an arrhythmia signal reflecting respiratory arrhythmia as the output signal.

The heart rhythm detection unit and the respiratory dynamics determination unit are thus advantageously connected to a unit for detecting respiratory arrhythmia, which based on the heart rate and respiratory dynamics, supplies a signal that corresponds to the respiratory arrhythmia and thus makes a further predictive parameter available for the implantable cardiac diagnostics.

Such a device offers the advantage of capturing an additional parameter for predicting imminent cardiac dysrhythmia, and/or cardiac decompensation (worsening heart failure predictor: WHF-P), and/or for VT/SVT discrimination, and/or for therapy control in implantable systems, without placing additional requirements on the electrode systems.

This results in an implantable device or module as a sensor for cardiac function diagnostics, based on the detection of respiratory arrhythmia.

At least one embodiment of the invention picks up the basic principle of respiratory sinus arrhythmia. This cardiorespiratory interaction forms part of the autonomous control of the heart rhythm. The hypothesis of at least one embodiment of the invention states that restricted or non-verifiable respiratory arrhythmia, at the juvenile or adult age, points to impaired or restricted autonomous control of the heart rhythm or is caused by acute cardiac decompensation. Because measuring the heart rate and respiratory dynamics is already established in electronic implants, implementing a corresponding correlation unit is also possible and promises a very robust parameter for assessing the autonomous control of the heart rhythm.

The respiratory dynamics detection unit is preferably configured to determine a respective moment of maximum inspiration and/or maximum expiration in a signal to be captured via the at least two electrode poles.

In addition or as an alternative, the respiratory dynamics detection unit can be configured to determine a phase of inspiration and/or expiration in a signal to be captured via the at least two electrode poles.

The respiratory dynamics detection unit is moreover preferably configured to determine a respiratory rate in a signal to be captured via the at least two electrode poles.

According to advantageous variants, the implantable electronic device can be an implantable cardioverter/defibrillator (ICD), an implant for cardiac resynchronization therapy (CRT) without (CRT-P) or with (CRT-D) defibrillator function, an implantable pulse generator (IPG), which is a cardiac pacemaker, for example, each being connected or to be connected to customary electrode lines. An implant for cardiac resynchronization therapy (CRT) is configured to additionally stimulate the left ventricle of a heart. Moreover, the implantable electronic device can also be configured as an implantable rhythm monitor (biomonitor) without integrated therapy function.

In all instances, the implantable electronic device is preferably configured to continuously, or at least regularly, for example once a day, determine the arrhythmia signal reflecting the respiratory arrhythmia.

The implantable electronic device preferably comprises a memory for receiving diagnostics parameters and is configured to store in the memory one or more parameters describing the arrhythmia signal reflecting the respiratory arrhythmia.

Especially in conjunction with the latter variant, it is advantageous for the implantable electronic device to additionally comprise a telemetry unit for transmitting data to a service center for further evaluation of the transmitted data, and to be designed, for this purpose, to transmit the parameter or parameters describing the arrhythmia signal reflecting the respiratory arrhythmia by means of the telemetry unit to a service center for further evaluation.

The evaluation unit is furthermore preferably configured to further evaluate the arrhythmia signal reflecting the respiratory arrhythmia for VT/sinus tachycardia discrimination so that it generates an output signal indicating ventricular tachycardia when the arrhythmia signal reflecting the respiratory arrhythmia drops below a threshold value, and a ventricle rhythm detected by a heart rhythm detection unit exceeds a threshold value.

Conversely, the evaluation unit is preferably configured to further evaluate the arrhythmia signal reflecting the respiratory arrhythmia for VT/sinus tachycardia discrimination so that it generates an output signal indicating sinus tachycardia when the arrhythmia signal reflecting the respiratory arrhythmia exceeds a threshold value, even if a ventricle rhythm detected by a heart rhythm detection unit exceeds a threshold value.

In both instances, it is advantageous for the device to comprise a therapy control unit, which is configured to control the delivery of antitachycardiac therapy. The evaluation unit can then be connected to the therapy control unit and control the delivery of antitachycardiac therapy so as to trigger the delivery of an antitachycardiac therapy when the arrhythmia signal reflecting the respiratory arrhythmia drops below a threshold value, and a ventricle rhythm detected by the heart rhythm detection unit exceeds a threshold value. The evaluation unit can also inhibit the delivery of an antitachycardiac therapy when the arrhythmia signal reflecting the respiratory arrhythmia exceeds a threshold value, even if a ventricle rhythm detected by the heart rhythm detection unit exceeds a threshold value.

### BRIEF DESCRIPTION OF THE DRAWINGS

The invention will now be described in more detail with reference to an exemplary embodiment illustrated the figures, wherein:

FIG. 1: shows a dual-chamber cardiac pacemaker and implantable cardioverter/defibrillator (ICD) as the implantable cardiac stimulator;

FIG. 2: shows several components of the cardiac stimulator in the form of a simplified block diagram;

FIG. 3: shows a triple-chamber cardiac pacemaker and implantable cardioverter/defibrillator (ICD) as the implantable cardiac stimulator;

FIG. 4: shows a section of a schematic block diagram of a module of a heart rhythm detection unit, a respiratory dynamics detection unit and an evaluation unit;

FIG. 5: is an example of respiratory sinus arrhythmia in the ECG;

FIG. 6: is an example of a correlation analysis;

FIG. 7: shows a schematic block diagram of an implantable device comprising a heart rhythm detection unit, a respiratory dynamics detection unit and an evaluation unit;

FIG. 8: shows a Sub-Q monitor; and

FIG. 9: shows a flow chart for SinT vs. VT discrimination.

### DETAILED DESCRIPTION OF THE INVENTION

The invention will be described below in greater detail by reference to an embodiment as an example, wherein the invention is not limited to this embodiment, but also comprises solutions, provided these solutions only embody the features of the independent claims.

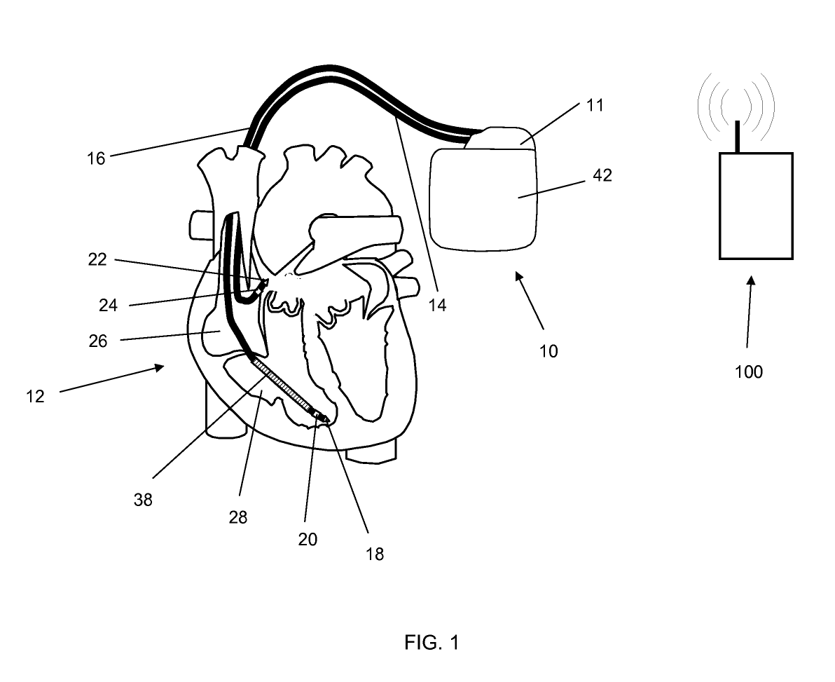

FIG. 1 shows a dual-chamber cardiac pacemaker and implantable cardioverter/defibrillator (ICD) as the implantable cardiac stimulator 10. This dual-chamber cardiac pacemaker is connected via electrode lines 14 and 16 to stimulation and sensing electrodes 22 and 24, and 20 and 18, respectively, in the atrium or ventricle of a heart and can thus deliver stimulation pulses to the heart and pick up electrical potentials from the heart.

Moreover, an external device 100 is shown in the vicinity of the implantable cardiac stimulator 10.

The electrode lines 14 and 16 are electrically connected to contact bushings in a header (connecting housing) 11 of the cardiac stimulator 10 via standardized plug connections. The electrode lines 14 and 16 are thus also connected to electronic components in an interior of a hermetically sealed metal housing 42 of the cardiac stimulator 10. These components are shown in more detail schematically below and determine the operating principle of the cardiac stimulator 10 according to at least one embodiment of the invention

The electrode line 14 is a right atrial electrode line with a distal end, wherein the distal end has an atrial tip electrode pole RA Tip 22 and, an atrial ring electrode pole RA Ring 24 located a short distance from RA Tip 22. Both electrodes are placed in the right atrium 26 of the heart 12.

The electrode line 16 is a right ventricular electrode line with a distal end, wherein the distal end has a right ventricular tip electrode pole RV Tip 18 and, a right ventricular ring electrode pole RV Ring 20 located in direct vicinity of RV Tip 18. Both electrodes are arranged in the apex of the right ventricle 28 of the heart 12.

Moreover, the right ventricular electrode line 16 includes a right ventricular shock coil RV Shock 38 in the form of a large-surface-area electrode pole for delivering defibrillation shocks.

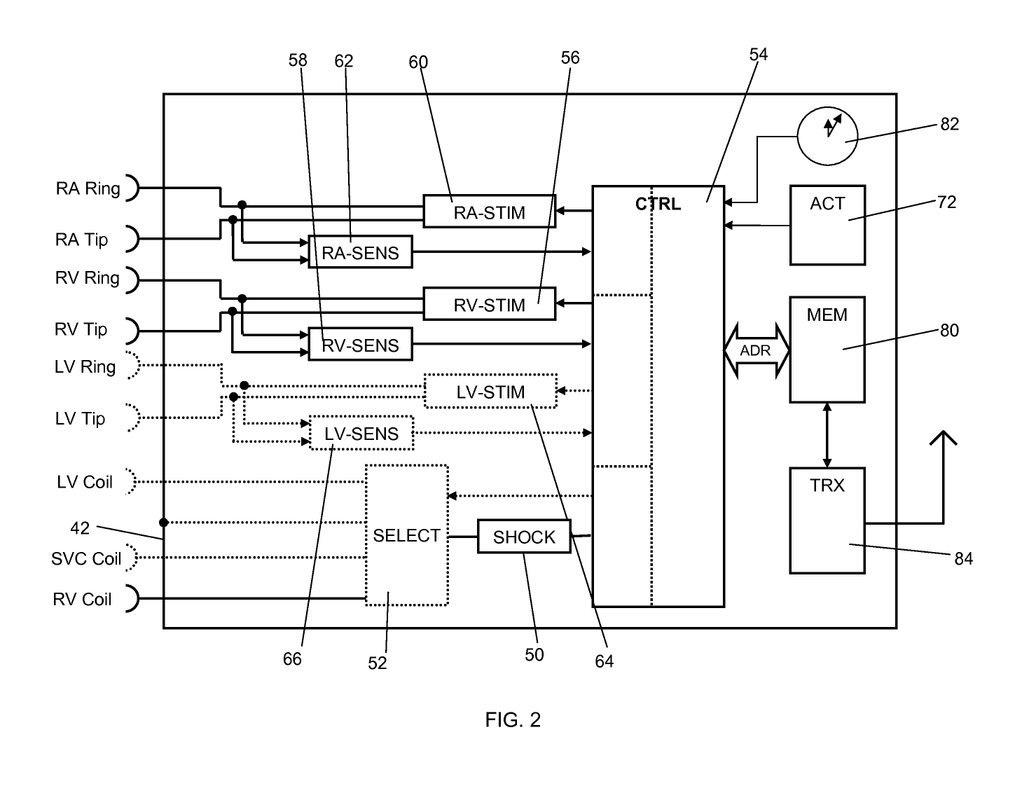

FIG. 2 shows the some of the key functional units of the cardiac stimulator 10. The dotted line shows further components as they can additionally be provided in an alternative variant of an implantable cardiac stimulator.

On the left side, the electrical connections for the different electrodes 18, 20, 22, 24 and 38 are shown. The shock electrode (shock coil) 38 is connected to a shock pulse generator 50. The shock pulse generator 50 is connected to a control unit 54, that controls the shock pulse generator 50 as needed for generating and delivering a cardioversion or defibrillation shock.

The connection for the right ventricular tip electrode pole RV Tip 18 and the connection for the right ventricular ring electrode pole RV Ring 20 are respectively connected to a right ventricular stimulation unit 56 and to a right ventricular sensing unit 58. Both the right ventricular stimulation unit 56 and the right ventricular sensing unit 58 are connected to the control unit 54.

The right ventricular stimulation unit 56 is configured to generate a right ventricular stimulation pulse in response to an actuation signal from the control unit 54, and to deliver the stimulation pulse to the right ventricular ring electrode pole and the right ventricular tip electrode pole via the connections. As an alternative, it is also possible for the housing 42 of the cardiac stimulator 10 to form a neutral electrode, for the right ventricular stimulation unit 56 to be connected to the connection for the right ventricular ring electrode pole RV Tip 18, and the housing 42 as another electrode for delivering a stimulation pulse. A right ventricular stimulation pulse differs from a defibrillation shock in that the stimulation pulse has significantly lower pulse intensity, so that differently from a defibrillation shock, it does not excite the entire myocardium of a ventricle all at once, but only the myocardial cells in the direct vicinity of the right ventricular tip electrode pole 18. This excitation then propagates over the entire ventricle as a result of natural stimulus conduction, thereby ensuring a stimulated contraction of the ventricle.

The right ventricular sensing unit 58 is configured to first amplify electrical potentials present at the connection of the right ventricular ring electrode pole RV Ring 20 and the right ventricular tip electrode pole RV Tip 18, by way of an input amplifier, and then filter the electrical potentials. The right ventricular sensing unit is further configured to evaluate curves of the electrical signals present at the inputs thereof, such that the right ventricular sensing unit 58 automatically detects a natural (intrinsic) contraction of the right ventricle. This can be done, for example, by comparing a curve of the signal present at the inputs of the right ventricular sensing unit 58 to a threshold value. Typically, the largest amplitude of the signal, the R-wave is characteristic of a natural contraction of the right ventricle, can be detected by a comparison to a threshold value. The right ventricular sensing unit 58 then emits a corresponding output signal to the control unit 54, the signal indicating a natural contraction of the right ventricle.

Analogously, the connection for the right atrial tip electrode pole and the connection for the right atrial ring electrode pole are connected both to a right atrial stimulation unit 60 and to a right atrial sensing unit 62, that are each in turn connected to the control unit 54. The right atrial stimulation unit 60 is configured to generate stimulation pulses, that are intense enough to excite the right atrial myocardium. The right atrial stimulation pulses can have different pulse intensities than the right ventricular stimulation pulses. The right atrial sensing unit 62 is configured to detect a P-wave from the curve of the differential signal present at the inputs, this wave characterizing a natural (intrinsic) contraction of the right atrium. If the right atrial sensing unit 62 detects such a P-wave, it generates an output signal and passes it on to the control unit 54, characterizing a natural contraction of the right atrium.

As another component of the cardiac stimulator 10, an activity sensor 72 is connected to the control unit 54. The activity sensor 72 is configured to capture a signal, for example a movement signal, dependent on the physical activity of a patient, and output a corresponding signal to the control unit 54 indicating the physical activity of the patient. This makes it possible for the control unit 54 to adapt the timing of the stimulation pulses to the need of the patient (hemodynamic need).

The cardiac stimulator 10 further comprises a memory unit 80 connected to the control unit 54, for storage of signals generated or evaluated by the control unit 54. In addition, the memory unit 80 stores control programs for the control unit 54 in modifiable form. Additionally, the control unit 54 is connected to a timer 84.

The cardiac stimulator 10 comprises at least one bidirectional telemetry interface 84, to transmit stored data from the electronic implant to an external device 100, and conversely to receive programming settings and therapy commands from the external device 100.

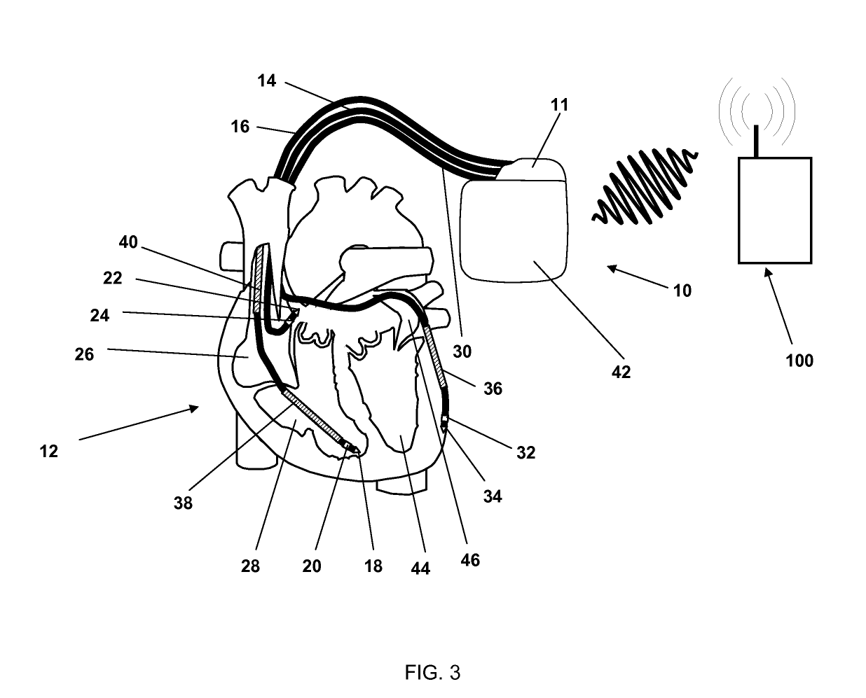

FIG. 3 shows an alternative variant of an implantable cardiac stimulator in the form of a biventricular triple-chamber cardiac stimulator/ICD 10′. This stimulator is connected via the connection block 11 (header) to a right ventricular electrode line 16, a right atrial electrode line 14 and, in addition, to a left ventricular electrode line 30.

These electrode lines are permanently implanted in the heart 12. To this end, the right ventricular electrode line 16 comprises, at the distal end, a bipolar stimulation and perception electrode comprising a tip electrode pole 18 and a ring electrode pole 20. Moreover, electrode line 16 is equipped with a distal shock coil 38 and a proximal shock coil 40. The distal shock coil 38 is arranged so that it is located in the right ventricle 28. The proximal shock coil 40 is located in the upper part of the right atrium 26. At the distal end, the atrial electrode line 14 comprises a bipolar stimulation and perception electrode, that is formed by a tip electrode pole 22 and a ring electrode pole 24, implanted in the right atrium 26. The distal end of the left ventricular electrode line 30 comprises a bipolar stimulation and perception electrode comprising a distal ring electrode pole 34, and a proximal ring electrode pole 32. The left ventricular electrode line 30 is further equipped with a shock coil 36.

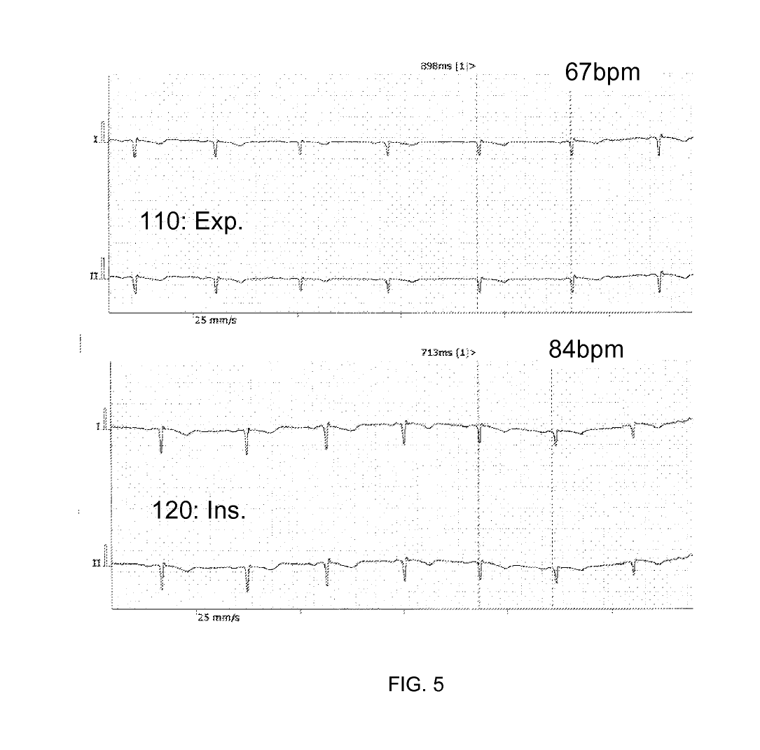

The electrode line 30 is a left ventricular electrode line with a distal end, wherein the distal end has a left ventricular tip electrode pole LV Tip 34, and a left ventricular ring electrode pole LV Ring 32 in the vicinity of LV Tip 34. In addition, the left ventricular electrode line 30 includes a left ventricular shock coil 36, which is not described in detail but shown in FIG. 5, for delivering defibrillation shocks to the left ventricle. This shock coil 36 is arranged so that it extends from the left ventricle 44 up to the left atrium 46. The electrically active housing 42 of the implant 10 represents another electrode for shock delivery.

The left ventricular electrode line 30 is conducted from the right atrium 26 of the heart 12 via the coronary sinus to a lateral vein branching off the coronary sinus and is therefore also referred to as a coronary sinus electrode line, or CS electrode line.

As is apparent from FIG. 2, based on the dotted components, the connection for the left ventricular tip electrode pole LV Tip and the connection for the left ventricular ring electrode pole LV Ring are also connected to a left ventricular stimulation unit 64 and a left ventricular sensing unit 66. The left ventricular stimulation unit 64 and the left ventricular sensing unit 66 are likewise connected to the control unit 54. Both function similarly to the above-described stimulation units 56 and 60 and the sensing units 58 and 62.

Via a connection LV-COIL and an electrode selection unit 52, the left ventricular shock coil 36 is likewise connected to the shock generator 50. Using the electrode selection unit 52, the control unit 54 can select two or more electrodes (including the conductive housing 42) to deliver a shock.

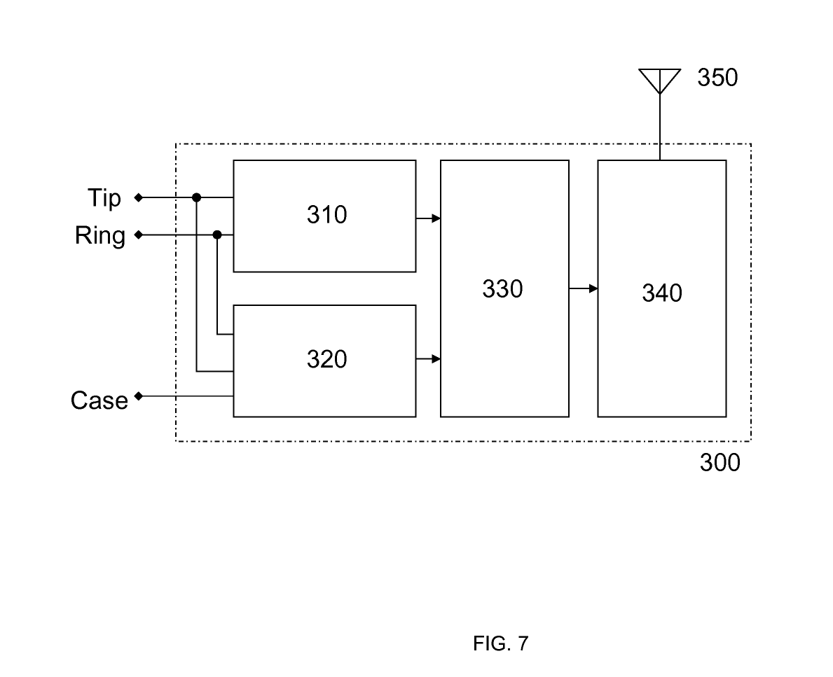

FIG. 2 does not show a heart rhythm detection unit 310 and a respiratory dynamics detection unit 320, however on the input side, detection units 310 and 320 are preferably connected to at least one tip electrode pole, for example the tip electrode pole RV Tip 18, and a ring electrode pole, for example the ring electrode pole RV Ring 20. Furthermore, on the output side, detection units 310 and 320 are connected to an evaluation unit 330. The heart rhythm detection unit 310, respiratory dynamics detection unit 320 and evaluation unit 330 can be part of the control unit 54. The heart rhythm detection unit 310, respiratory dynamics detection unit 320 and evaluation unit 330 can also be implemented as a separate implantable device (see FIG. 7) or be part of another implantable device different from the implantable device shown in FIGS. 1 to 3.

Electrical signals representing an intracardiac electrocardiogram (ECG) can be picked up via the individual at least two electrode poles.

FIG. 7 shows a possible block diagram of a module or a device for detecting respiratory arrhythmia. Within the scope of this description, such a device or module is also referred to as a sensor for detecting respiratory arrhythmia. The implantable device (or the module thereof) comprises a heart rhythm detection unit 310, connected to two implanted electrode poles (tip, ring), which serve as ECG or IEGM leads. The implant further comprises an impedance-based respiratory dynamics detection unit 320, connected to the ECG/IEGM electrode poles (tip, ring) and additionally comprises a further connection to the implant housing (case). Tripolar impedance measurement can thus be employed for measuring the breathing cycle. The measuring current is fed via the tip electrode pole and the housing (case 42) of the device and a voltage measurement is conducted via a ring electrode pole and the housing (case). This allows the breathing cycle and hence the time of maximum inspiration and maximum expiration to be detected.

A correlation analysis unit 330 serves as an evaluation unit for detecting any existing respiratory sinus arrhythmia. Correlation analysis unit 330 is connected to the heart rhythm detection unit 310 and to the respiratory dynamics detection unit 320. The result of the correlation analysis is then made available to a diagnostics and/or telemetry and/or therapy control unit 340 and optionally transmitted via a telemetry interface 350.

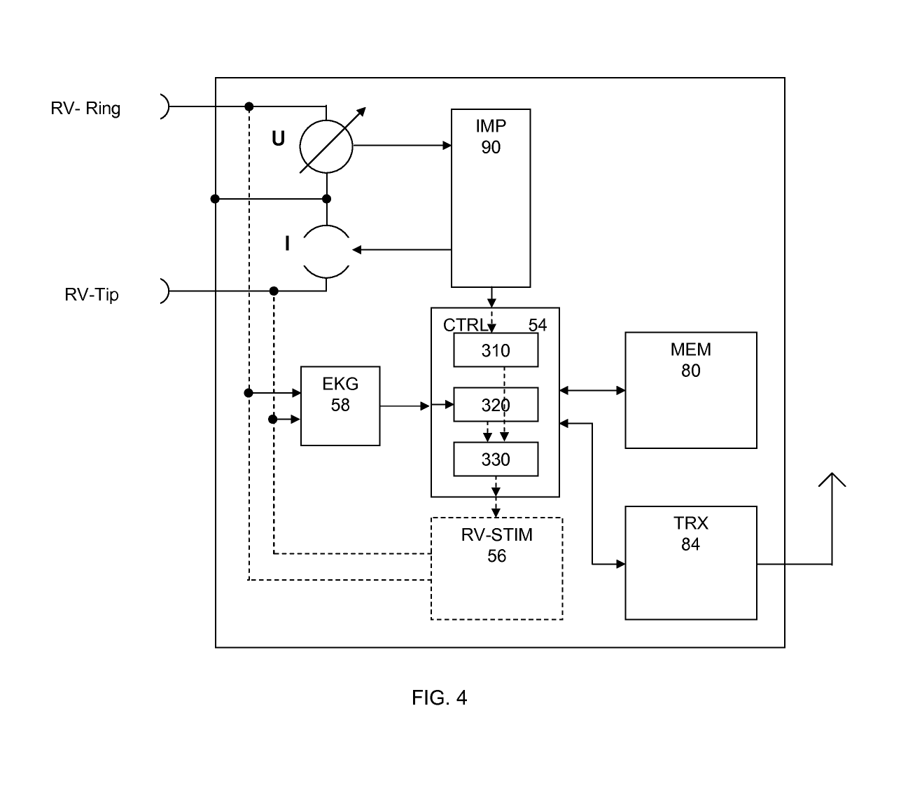

FIG. 4 shows schematically how a module for detecting respiratory arrhythmia can be integrated in a cardiac therapy device as shown in FIGS. 1 to 3. For this purpose, only the components required for detecting respiratory arrhythmia are shown in FIG. 4. It is apparent that the heart rhythm detection unit 310, the respiratory dynamics detection unit 320, as well as the correlation analysis unit 330 that serves as the evaluation unit, are implemented as part of the control unit 54. The therapy control unit 340 (not explicitly shown in FIG. 4) is also implemented by the control unit 54. FIG. 4 furthers shows a constant current source I, a voltage measurement unit U, as well as an impedance determination unit 90, as components connected upstream of the respiratory dynamics detection unit 320. The constant current source generates short, biphasic constant current pulses. In the example shown in FIG. 4, the heart rhythm detection unit 310 is connected to the ventricular sensing unit 58 to receive an intracardiac ECG (IECG).

FIG. 5 shows an ECG recording of a subject with pronounced respiratory sinus arrhythmia. The upper ECG (reference numeral 110) shows the heart rhythm with a maximum expiration at a heart rate of 67 bpm. The lower ECG (reference numeral 120) shows the heart rate risen to 84 bpm following maximum inspiration. Both recordings were taken immediately after each other under unchanged conditions (subject sitting down, resting).

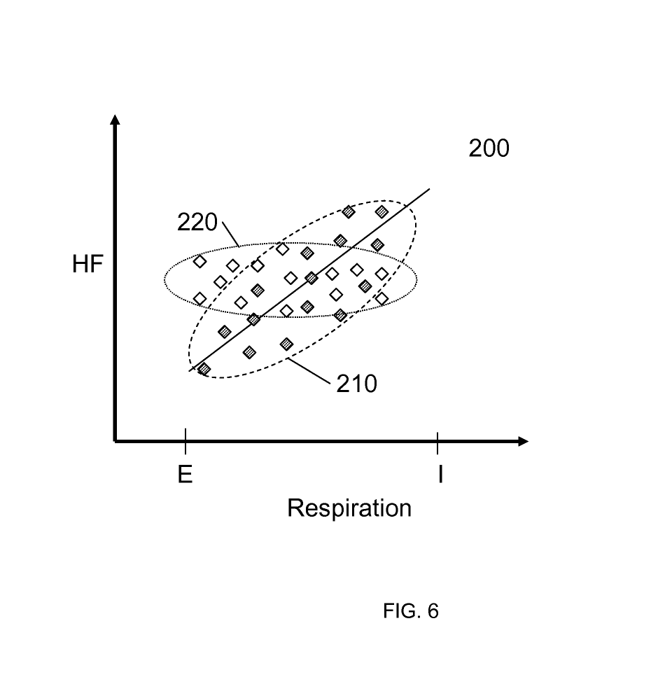

FIG. 6 shows the relationship (reference number 200) between respiration and heart rate, as between respiratory dynamics and heart rhythm. Respiration is characterized by maximum expiration (E) and maximum inspiration (I). The heart rate (HF) is shown as depending on respiration.

In a patient with a healthy heart, a good correlation can be shown between respiration and heart rate (reference numeral 210). In a patient with cardiac insufficiency, in contrast, no correlation can be shown between respiration and heart rate (reference numeral 220). This differentiating feature is supposed to be captured by the solution according to the invention and utilized for characterizing the cardiac state. Preferably, the evaluation unit 330 utilizes the relationship as shown in FIG. 6.

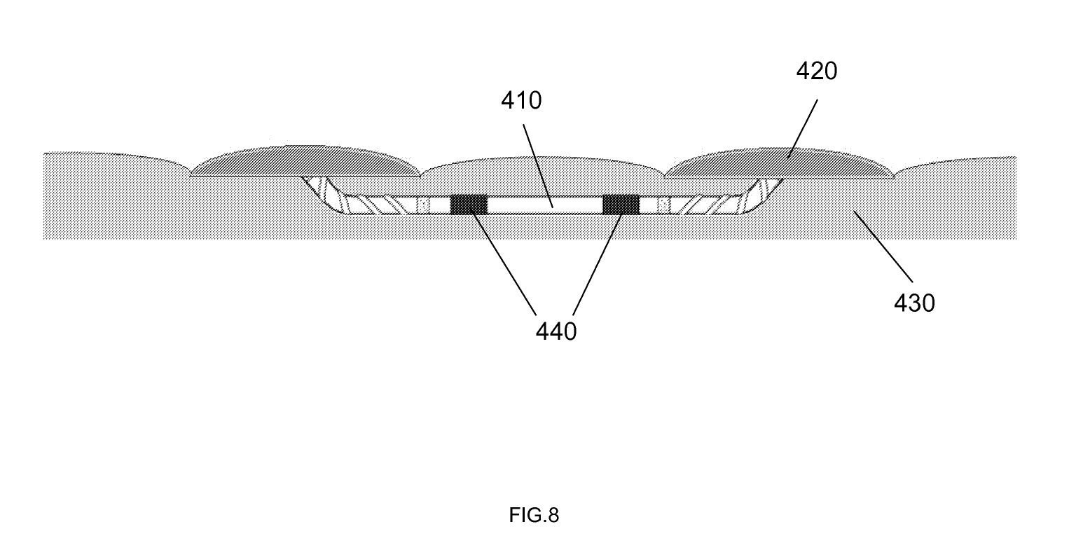

FIG. 8 shows another embodiment of the sensor for respiratory arrhythmia. It includes a semi-implantable system implanted subcutaneously 430, comprising an implanted electrode system 410, fixation units 420 attached to the body surface, and also contains active electronics and a power source. The electrode system is configured to be implanted in an epithelialized channel, or forms an epithelialized channel. In this embodiment, the heart rate detection and impedance-based respiratory dynamics measurements are carried out by the two electrode poles 440 as shown in FIG. 8.

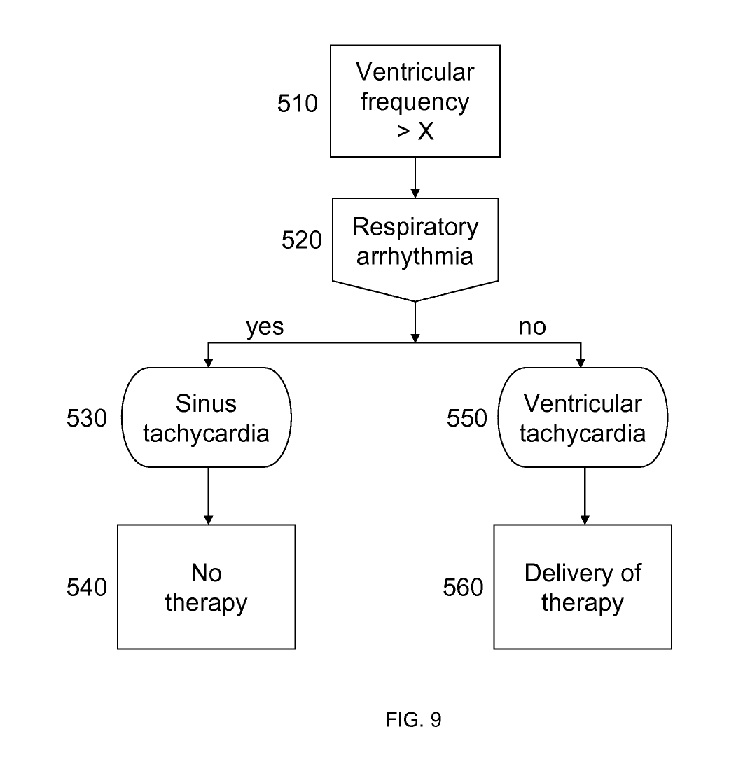

FIG. 9 shows a further use of the sensor for respiratory sinus arrhythmia. The flow chart shows the possible application for discrimination between stress-induced sinus tachycardia and ventricular tachycardia (VT).

If the patient has verifiable respiratory sinus arrhythmia, the system can additionally check whether this respiratory sinus arrhythmia can continued to be verified (32) with a fast ventricular rhythm (510). If this is the case (530), a stress-induced sinus frequency increase (530) can be assumed and antitachycardiac therapy can be inhibited (540). If the respiratory sinus arrhythmia disappears with increased ventricular frequency, ventricular tachycardia (550) can be assumed and an antitachycardiac therapy (560) should be initiated.

An advantage of the device according to at least one embodiment of the invention is that the detection of the parameter indicating respiratory arrhythmia does not place any additional demands on the electrode systems, and can be implemented with existing implant platforms.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. An implantable electronic device or device module, comprising
a heart rhythm detection unit;
a respiratory dynamics detection unit;
an evaluation unit;
at least two electrode poles;
at least one connection for the at least two electrode poles configured to sense electrical potentials; wherein the at least two electrode poles are connected to the heart rhythm detection unit and are configured to capture a signal that represents a heart rhythm signal, and wherein the heart rhythm detection unit is configured to supply said heart rhythm signal as an output signal;
wherein the at least two electrode poles are further connected to the respiratory dynamics determination unit and are further configured to capture a respiratory dynamics signal that represents respiratory dynamics, and wherein the respiratory dynamics detection unit is configured to supply the respiratory dynamics signal as the output signal;
wherein the heart rhythm detection unit and the respiratory dynamics detection unit are connected to the evaluation unit; and,
wherein the evaluation unit is configured to
evaluate the heart rhythm signal in conjunction with a temporally associated respective respiratory dynamics signal, and
generate an arrhythmia signal that reflects respiratory arrhythmia as the output signal.

2. The implantable electronic device according to claim 1, wherein the evaluation unit is further configured to generate an arrhythmia signal that represents a correlation between the heart rhythm signal and a temporally associated respective arrhythmia signal as the output signal.

3. The implantable electronic device according to claim 1, wherein the respiratory dynamics detection unit is further configured to determine a respective moment of maximum inspiration and/or maximum expiration in the signal that represents a heart rhythm signal.

4. The implantable electronic device according to claim 1, wherein the respiratory dynamics detection unit is further configured to determine a phase of inspiration and/or expiration in the signal that represents a heart rhythm signal.

5. The implantable electronic device according to claim 1, wherein the respiratory dynamics detection unit is further configured to determine a respiratory rate in the signal that represents a heart rhythm signal.

6. The implantable electronic device according to claim 1, further comprising electrode lines, wherein the implantable electronic device is an ICD, a CRT-D, a CRT-P or an IPG, and is connected to the electrode lines.

7. The implantable medical device according to claim 1, wherein the implantable electronic device is an implantable rhythm monitor.

8. The implantable electronic device according to claim 1, configured to continuously, or at least once a day, determine the arrhythmia signal that reflects the respiratory arrhythmia.

9. The implantable electronic device according to claim 1, further comprising a memory configured to receive diagnostics parameters, wherein the implantable electronic device is configured to store, in the memory, one or more parameters that describe the arrhythmia signal that reflects the respiratory arrhythmia.

10. The implantable electronic device according to claim 9, further comprising a telemetry unit configured to transmit data to a service center to further evaluate the transmitted data; and,
wherein the transmitted data comprises the one or more parameters that describe the arrhythmia signal that reflects the respiratory arrhythmia.

11. The implantable electronic device according to claim 1, wherein the evaluation unit is further configured to
evaluate the arrhythmia signal that reflects the respiratory arrhythmia for VT/sinus tachycardia discrimination; and,
generate an output signal that indicates ventricular tachycardia when the arrhythmia signal that reflects the respiratory arrhythmia drops below a first threshold value, and a ventricle rhythm detected using the heart rhythm detection unit exceeds a second threshold value.

12. The implantable electronic device according to claim 11, further comprising a therapy control unit;
wherein the evaluation unit is connected to the therapy control unit, and is further configured to
trigger the delivery of an antitachycardiac therapy from the therapy control unit when the arrhythmia signal that reflects the respiratory arrhythmia drops below the first threshold value, and when a ventricle rhythm detected using the heart rhythm detection unit exceeds the second threshold value.

13. The implantable electronic device according to claim 1, wherein the evaluation unit is further configured to
evaluate the arrhythmia signal that reflects the respiratory arrhythmia for VT/sinus tachycardia discrimination; and,
generate an output signal that indicates sinus tachycardia when the arrhythmia signal that reflects the respiratory arrhythmia exceeds a first threshold value, and when a ventricle rhythm detected using the heart rhythm detection unit exceeds a second threshold value.

14. The implantable electronic device according to claim 13, further comprising a therapy control unit, wherein the evaluation unit is connected to the therapy control unit and is further configured to inhibit the delivery of an antitachycardiac therapy from the therapy control unit when the arrhythmia signal that reflects the respiratory arrhythmia exceeds the first threshold value, and when a ventricle rhythm detected using the heart rhythm detection unit exceeds the second threshold value.
